(19) United States
(12) Patent Application Publication
(10) Pub. No.: US 2022/0218584 A1
(43) Pub. Date: Jul. 14, 2022

(54) TOPICAL COMPOSITION AND METHOD OF USE

(71) Applicant: Allergan Sales, LLC, North Chicago, IL (US)

(72) Inventors: Patrick Nido, Brea, CA (US); Kuniko Kadoya, San Diego, CA (US); Prithwiraj Maitra, Ladera Ranch, CA (US); Rahul Mehta, San Marcos, CA (US); Elizabeth Makino, Carlsbad, CA (US)

(73) Assignee: ALLERGAN SALES, LLC, North Chicago, IL (US)

(21) Appl. No.: 17/574,893

(22) Filed: Jan. 13, 2022

Related U.S. Application Data
(60) Provisional application No. 63/136,892, filed on Jan. 13, 2021.

(51) Int. Cl. A61K 8/64; A61Q 19/08; A61K 8/9728; A61K 8/9722; A61K 8/9789; A61K 8/89; A61K 8/81

(52) U.S. Cl. CPC A61K 8/64; A61Q 19/08; A61K 8/9728; A61K 8/8147; A61K 8/9789; A61K 8/89; A61K 8/9722

(57) ABSTRACT

The present invention relates to a topical composition that can be used to treat skin aging of the neck and décolletage area.

### CROSS-REFERENCE TO RELATED APPLICATION

This application claims priority under 35 U.S.C. § 119(e) to U.S. Provisional Application Ser. No. 63/136,892, filed on Jan. 13, 2021, the disclosure of which is incorporated herein by reference in its entirety.

### FIELD OF THE INVENTION

The present invention relates to topical compositions and methods for skincare treatment employing the topical compositions disclosed herein. More specifically, the topical compositions and methods for skincare disclosed herein are cosmetic formulations, particularly anti-aging cosmetic formulations, that may be applied to various areas on humans skin, particularly the neck and décolletage area.

### BACKGROUND

When skin ages naturally or prematurely, it thins and gradually loses its firmness, wrinkles and/or sags. This can be explained by the fact that the elastic fibers of the dermal extracellular matrix, forming the support and conferring elasticity and strength to the skin are destroyed and become rare with age.

The face is the primary concern for most patients seeking skin rejuvenation treatments, however, other body parts such as the neck and décolletage area are significant target areas. During the process of aging, the neck experiences several anatomical changes including loss of elasticity, platysmal banding and fat accumulation. As the most superficial layer of the neck, the skin bears the cumulative burden of years of sun exposure resulting in loss of elasticity and firmness and the appearance of horizontal skin rhytides due to the degeneration of dermal collagen and elastic fibers. This is also true for the décolletage area where typical aesthetic concerns include appearance of uneven skin tone, hyperpigmentation and rough skin texture.

Neck skin is more flexible and extensible than facial skin to allow for head movements and experiences a greater loss in elasticity with aging; it is also thinner and more delicate and therefore more damage prone. Due to lower sebum secretion, neck skin shows more severe aging patterns and deeper wrinkles than facial skin. Furthermore, neck skin has fewer appendages, which can delay post-procedural healing and outcome.

There is a need for compositions that are useful in counteracting the appearance of horizontal lines, wrinkles, crepey-like skin, hyperpigmentation (or skin tone unevenness) and sagging especially in the neck and décolletage area that is triggered by aging, dehydration, and other environmental factors.

### SUMMARY OF THE INVENTION

The present invention is a topical composition that may be applied to various skin areas of a human subject, particularly the neck and décolletage area. The topical composition comprises a tripeptide, a tetrapeptide or a combination thereof. In certain embodiments, the topical composition may further comprise one or more botanical extracts that are extracellular matrix (“ECM”) agents; cellular recycling (“CR”) agents; muscle contracting (“MR”) agents; antioxidants or combinations thereof.

In certain embodiments, the topical composition comprises about 0.0001 wt % to about 2.00 wt % based on the total weight of the composition of a tripeptide, about 0.0001 wt % to about 2.00 wt % based on the total weight of the composition of a tetrapeptide or a combination thereof.

In certain embodiments, the topical composition will comprise about 0.001 wt % to about 5 wt % based on the total weight of the composition of one or more botanical extracts wherein the one or more botanical extracts comprise one or more ECM agents; one or more CR agents; one or more MR agents; one or more antioxidants or combinations of the foregoing.

The topical composition may be a solution, suspension, dispersion, emulsion, gel, cream, lotion, ointment or serum and further comprise conventional topical/cosmetic carriers and excipients such as one or more emollients, thickeners/viscosity enhancing agents, film forming polymers, chelating agents, humectants, preservatives, pH adjusting agents, buffering agents, solvents, surfactants, emulsifiers, sunscreen agents and combinations of the foregoing.

The topical composition may be applied to various skin areas of a human subject, particularly the neck and décolletage area at least once or more times a day, preferably two, three or four times a day.

The topical compositions are particularly useful as anti-aging treatments and will reduce or improve the appearance of horizontal lines, wrinkles, crepey-like skin, hyperpigmentation, redness, and sagging especially in the neck and décolletage area triggered by aging, dehydration, and other environmental factors.

### DETAILED DESCRIPTION OF THE DRAWINGS

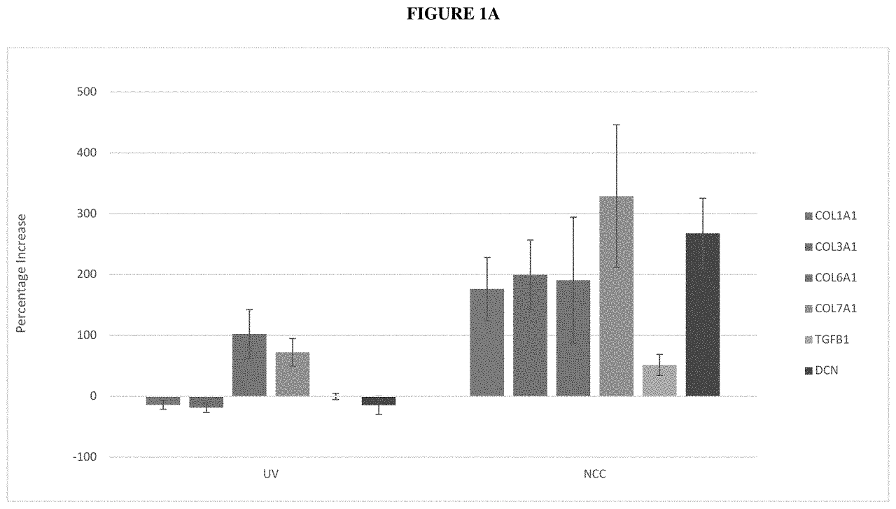

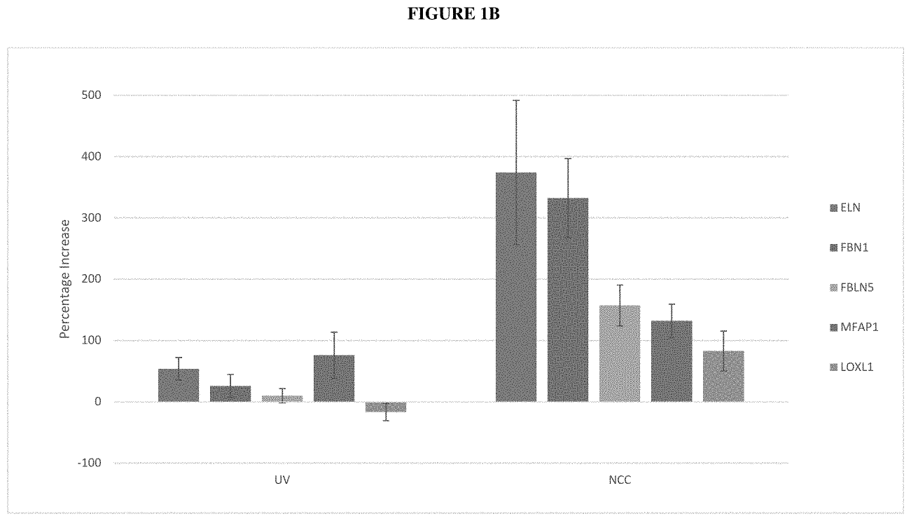

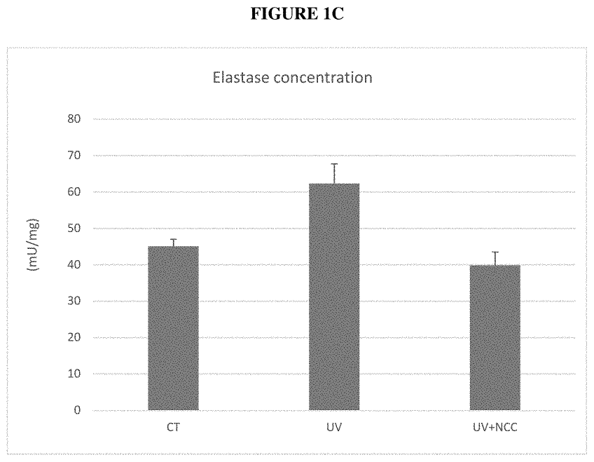

FIGS. 1A-1C show the in vitro extracellular matrix results of the tests described in Example 3.

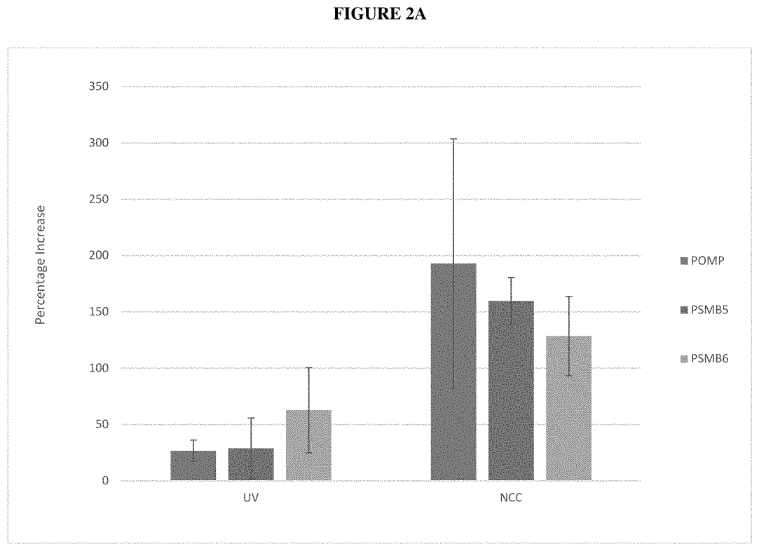

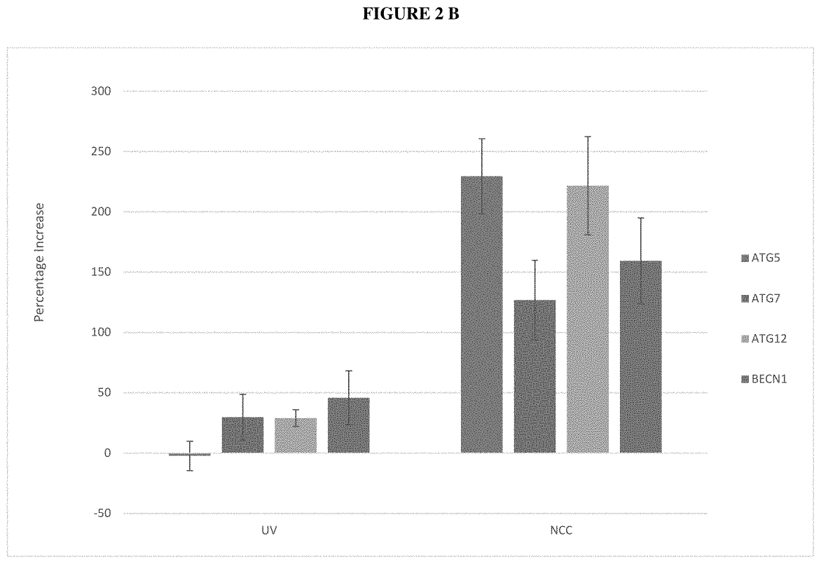

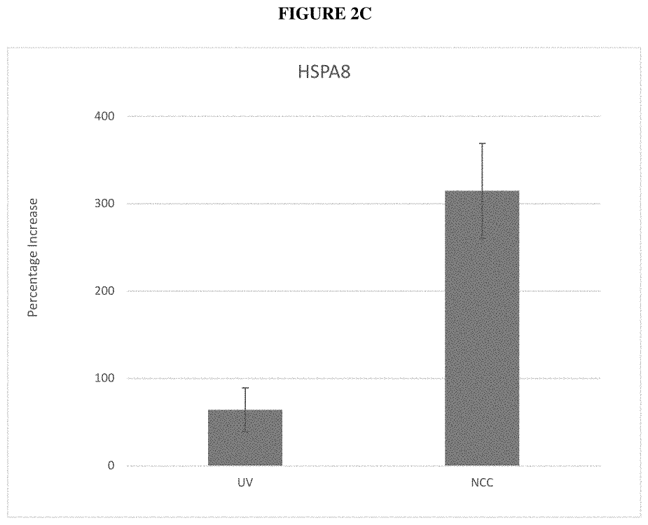

FIGS. 2A-2C show the in vitro protein recycling and matrix enzyme results of the tests described in Example 3.

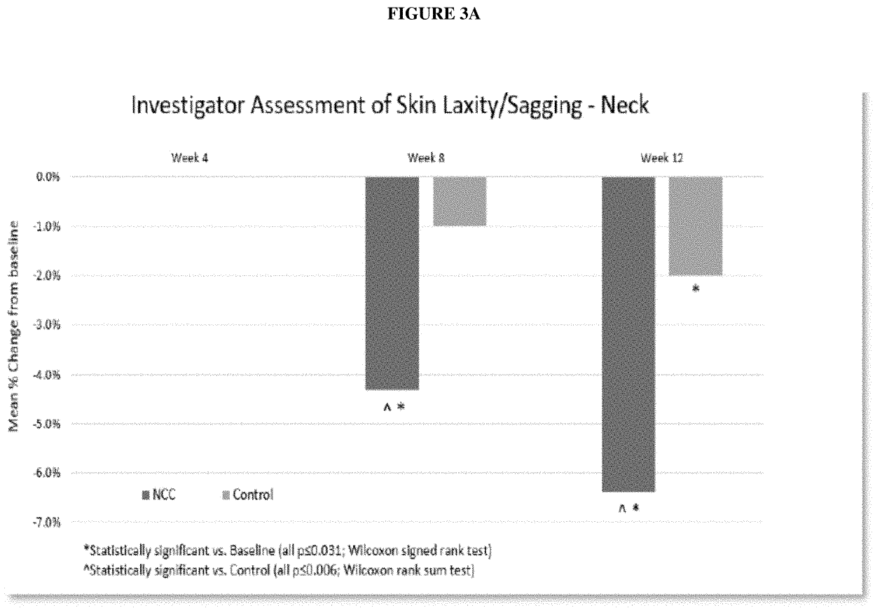

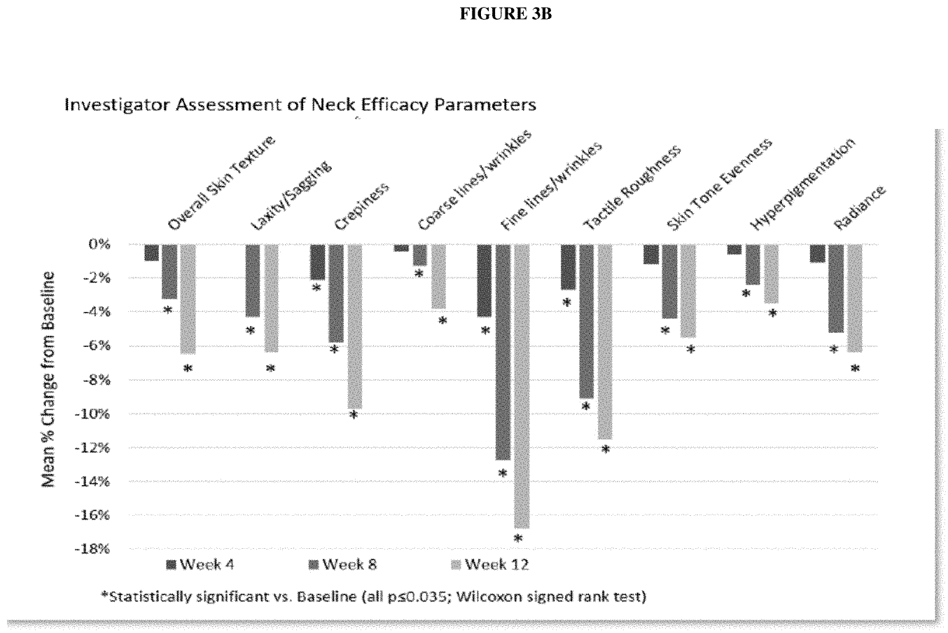

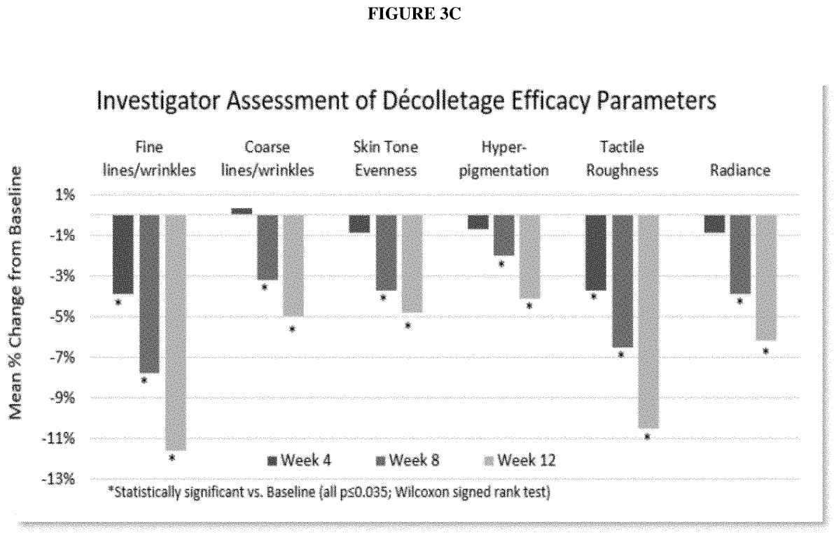

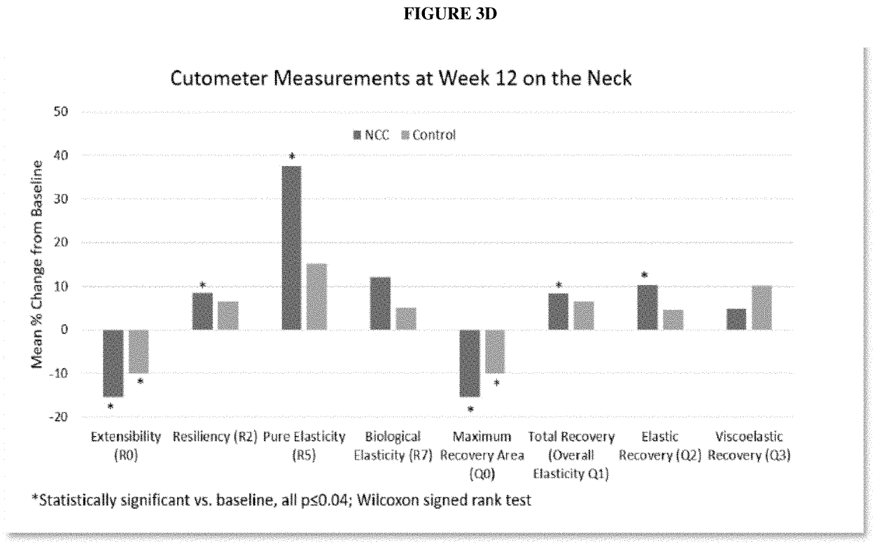

FIGS. 3A-3D shows the results of the clinical tests described in Example 4.

### DETAILED DESCRIPTION OF THE INVENTION

Before the present invention is further described, it is to be understood that this invention is not limited to the particular embodiments described. It is also to be understood that the terminology used herein is for the purpose of describing particular embodiments only, and is not intended to be limiting.

It should be noted that as used herein, the singular forms “a,” “an,” and “the” include plural referents unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the terms “extracellular matrix agent” or “ECM agent” refers to a compound, preferably a botanical extract that has a composition similar to the main components of the skin dermis and/or dermoepidermal junction (“DEJ”) such collagen, elastin and laminins. The ECM agents may increase ECM synthesis and/or inhibit ECM enzymes such as collagenases and elastases that catalyze the degradation or breakdown of collagen and elastin fibers. The ECM agent may also reduce carbamylation that in turn prevents or deters the deterioration of collagen and other EMC proteins and thereby improves the organization of collagen.

As used herein, the terms “cellular recycling agent” or “CR agent” refers to a compound, preferably a botanical extract that induces or promotes collagen recycling. For example, the RC agent may promote Endo180, an endocytic recycling glycoprotein expressed on fibroblasts that induces the collagen sustainability cycle or autophagy. The CR agent may also induce or maintain proteasome activity. For example, the CR agent may help maintain or increase ubiquitin-proteasome (“Ub-P”) levels and aid in decreasing senescence markers.

As used herein the term “muscle contracting agent” or “MR agent” refers to a compound, preferably a botanical extract that reduces or impedes muscle contraction in the target area or application site.

As used herein the term antioxidant broadly refers to a compound that retards oxidation or degradation. The antioxidant may retard the oxidation or degradation of one or more ingredients in the topical composition and/or may retard the oxidation or degradation of the components of the skin cells in the target area or application site. Unless specifically stated, the antioxidant may be a commonly known antioxidant such as those described in the United State Pharmacopeia or Handbook of Pharmaceutical Excipients, a botanical extract or a combination thereof.

Unless defined otherwise, all technical and scientific terms used herein have the same meaning as commonly understood by one of ordinary skill in the art to which this invention belongs. For example, as used herein the following nomenclature maybe employed to refer to various amino acids used of the peptides useful in the present invention: Alanine (also referred to herein as “Ala” or “A”), Arginine (also referred to herein as “Arg” or “R”), Asparagine (also referred to herein as “Asn” or “N”), Aspartic acid (also referred to herein as “Asp” or “D”), Cysteine (also referred to herein as “Cys” or “C”), Glutamic acid (also referred to herein as “Glu” or “E”), Glutamine (also referred to herein as “Gln” or “Q”), Glycine (also referred to herein as “Gly” or “G”), Histidine (also referred to herein as “His” or “H”), Isoleucine (also referred to herein as “Ile” or “I”), Leucine (also referred to herein as “Leu” or “L”), Lysine (also referred to herein as “Lys” or “K”), Methionine (also referred to herein as “Met” or “M”), Phenylalanine (also referred to herein as “Phe” or “F”), Proline (also referred to herein as “Pro” or “P”), Serine (also referred to herein as “Ser” or “S”), Threonine (also referred to herein as “Thr” or “T”), Tryptophan (also referred to herein as “Trp” or “W”), Tyrosine (also referred to herein as “Tyr” or “Y”), Valine (also referred to herein as “Val” or “V”).

Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The present invention is a topical composition that may be applied to a human subject's outer skin surface. Although the topical composition may be applied to any skin surface, such as face, arms, legs, or torso, in certain embodiments the preferred or target skin surface for application is the neck and décolletage area of the subject.

The topical compositions in accordance with the present invention may a solution, suspension, dispersion, emulsion, gel, cream, lotion, ointment or serum. In certain embodiments, the composition is a gel, cream, lotion or ointment. Embodiments of the topical compositions may exhibit a viscosity of less than 100,000 cps, preferably less than 75,000 cps and more preferably less than 50,000 cps at 25° C. when tested using a conventional viscosity apparatus, such as a Brookfield RVT viscometer with spindle 2 and 20 rpms. In certain embodiments, the topical composition will exhibit a viscosity at 25° C. between about 3,000 cps and 75,000 cps, preferably about 5,000 cps and about 60,000 cps and more preferably about 7,000 cps and about 50,000 cps.

The topical compositions in accordance with the present invention should also exhibit a pH of about 5 to about 8, preferably about 5.5 to about 7.5 and more preferably about 6 to about 7. The pH may be controlled using buffer solutions or other pH modifying agents. Examples of pH modifying agents include but are not limited to, phosphoric acid and/or phosphate salts, citric acid and/or citrate salts, hydroxide salts (i.e., calcium hydroxide, sodium hydroxide, potassium hydroxide) and amines, such as triethanolamine. Examples of buffers include, acetic acid and salts, citric acid and salts, boric acid and salts, and phosphoric acid and salts such as mono- and dipotassium phosphate, citric acid/sodium citrate, and dibasic sodium phosphate/citric acid.

The topical compositions in accordance with the present invention may comprise one or more tripeptides. In certain embodiments, the topical compositions will comprise about 0.0001 wt % to about 2.00 wt %, preferably about 0.0002 wt % to about 1.00 wt % and more preferably about 0.0003 wt % to about 0.05 wt % of the total weight of the topical composition of one or more tripeptides. The tripeptide should reduce progerin production in the skin cells. Progerin is a protein that causes nuclear defects and can increase DNA damage. Examples of tripeptides that may be used in the compositions are described in U.S. Pat. No. 10,286,030 and U.S. Patent Application Publication No. 2012/0076842, which are incorporated herein by reference. Additional examples, include but are not limited to, palmitoyl tripeptide-1 (commercially available under the tradename (MATRIXYL 3000), palmitoyl tripeptide-5 (commercially available under the tradename SYN-COLL), copper tripeptide-1 (commercially available under the tradename TEGO PEP3-RECOVER), palmitoyl tripeptide-28 (commercially available under the tradename ECM MODULINE PEPTIDE POWDER), tripeptide-1 (commercially available under the tradename (KOLLAREN) and trifluoroacetyl tripeptide-2 (commercially available under the tradename PROGELINE). In certain embodiments, the tripeptide may be functionalized with known chemical moieties such as a fatty acid to improve the properties of the tripeptide such as to enhance the skin permeation or penetration. In certain embodiments, the preferred tripeptide comprises Val-Try-Val amino acid sequence.

The topical compositions in accordance with the present invention may comprise one or more tetrapeptides. In certain embodiments, the topical composition will comprise about 0.0001 wt % to about 2.00 wt %, preferably about 0.0002 wt % to about 1.00 wt % and more preferably about 0.0003 wt % to about 0.05 wt % of the total weight of the topical composition of one or more tetrapeptides. The tetrapeptide should enhance the natural elements that maintain collagen levels and elastin fiber assembly. The tetrapeptide may also induce the elastic fiber proteins, elastin and fibrilli-1 protein synthesis, assist in the synthesize of other proteins for elastic fiber assemblies such as fubulin5 and LOXL-1 and may assist with production of focal adhesion molecules such as talin and zyxin. Examples of tetrapeptides that may be used in the topical compositions are described in U.S. Pat. No. 10,286,030 and U.S. Patent Application Publication No. 2012/0076842, which are incorporated herein by reference. Additional examples include but are not limited to palmitoyl tetrapeptide-7 (commercially available under the tradenames MATRIXYL 3000 or HALOXYL), tetrapeptide-30 (commercially available under the tradename TEGO PEP 4-EVEN), palmitoyl tetrapeptide-72 (commercially available under the tradename SKINARCH) and acetyl tetrapeptide-2 (commercially available under the tradename UPLEVITY). In certain embodiments, the tetrapeptide may be functionalized with known chemical moieties such as fatty acids to improve the properties of the tripeptide such as to enhance the skin permeation or penetration. In certain embodiments, the preferred tetrapeptide comprises Lys-Asp-Val-Try amino acid sequence.

Although other peptides may also optionally be present in the topical compositions of the present invention such as dipeptides, pentapeptide, hexapeptides and heptapeptides which are also described in U.S. Pat. No. 10,286,030 and U.S. Patent Application Publication No. 2012/0076842, in certain embodiments of the present invention, the topical compositions are free, (i.e., 0 wt % or below detectable limits) or substantially free, (i.e., less than 0.00005 or 0.00001 wt %) of peptides other than the one or more tripeptides, one or more tetrapeptides or a combination thereof.

The topical compositions in accordance with the present invention may comprise one or more ECM agents, preferably one or more botanical ECM agents. In certain embodiments, the topical compositions will comprise about 0.001 wt % to about 2.00 wt %, preferably about 0.005 wt % to about 1.50 wt % and more preferably about 0.007 wt % to about 1.00 wt % based on the total weight of the topical composition of one or more ECM agents. Examples of ECM agents that may be used in the present invention include, but are not limited, to Chlorella vulgaris extract as described in International Patent Application No. WO 2007/078056 and commercially available under the tradename CHLORELLAGEN, and mushroom extracts such as Lentinus edodes extract, commercially available under the tradename ACTIFICOL.

The topical compositions in accordance with the present invention may comprise one or more CR agents, preferably one or more botanical CR agents. In certain embodiments, the composition will comprise about 0.0005 wt % to about 2.00 wt %, preferably about 0.001 wt % to about 1.50 wt % and more preferably about 0.0025 wt % to about 1.00 wt % based on the total weight of the topical composition of one or more CR agents. Examples of CR agents that may be used in the present invention include but are not limited to Melissa officinalis leaf extract, aka lemon balm, commercially available under the tradename RECYCOLL 180 and rice extracts such a hydrolyzed rice protein, commercially available under the tradename PROLIXIR-ICE.

The topical compositions in accordance with the present invention may comprise one or more MR agents, preferably one or more botanical MR agents. In certain embodiments, the composition will comprise about 0.005 wt % to about 2.00 wt %, preferably about 0.01 wt % to about 1.50 wt % and more preferably about 0.05 wt % to about 1.00 wt % based on the total weight of the topical composition of one or more MR agents. Preferably, the botanical MR agent will comprise spilanthol. An example of an MR agent that may be used in the present invention includes, but is not limited to, Acmella oleacera extract, commercially available under the tradename GATULINE EXPRESSION.

The topical compositions in accordance with the present invention may comprise one or more antioxidants. In certain embodiments the topical composition will comprise about 0.0005 wt % to about 4.00 wt %, preferably about 0.0007 wt % to about 3.0 wt % and more preferably about 0.001 wt % to about 2.00 wt % based on the total weight of the topical composition of one or more antioxidants. Non-limiting examples of antioxidants that can be used with the topical compositions of the present invention include acetyl cysteine, ascorbic acid polypeptide, ascorbyl dipalmitate, ascorbyl methylsilanol pectinate, ascorbyl palmitate, ascorbyl stearate, butylated hydroxyanisole (BHA), butylated hydroxytoluene (BHT), t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, Helianthus annus (sunflower) seed oil, hydroquinone, isooctyl thioglycolate, kojic acid, Laminaria digitata extract, magnesium ascorbate, magnesium ascorbyl phosphate, methylsilanol ascorbate, natural botanical anti-oxidants such as Camellia sinensis green tea extract or Vitris vinifera (grape) callous culture extract, nordihydroguaiaretic acid, octyl gallate, phenylthioglycolic acid, potassium ascorbyl tocopheryl phosphate, potassium sulfite, propyl gallate, quinones, rosmarinic acid, Rosa moschata seed oil, Rosmarinus officinalis (rosemary) leaf extract, sodium ascorbyl phosphate, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, Tremella fuciformis (mushroom) extract, tris(nonylphenyl)phosphite, and Zea mays (corn) oil. In certain embodiments, the topical compositions comprise one or more antioxidants that are botanical extracts such as Physalis pubescens fruit juice, commercially available under the tradename PHYSAVIE-CHI, Dunaliela salina extract commercially available under the tradename IBR CLC, Polygonium aviculare extract commercially available under the tradename ELIX-IR. In certain embodiments, the topical compositions may also comprise one or more non-botanical extract antioxidants such as tocopherol, propyl gallate, hydroxyacetaphenone.

The topical compositions of the present invention may be a solution, suspension, dispersion, emulsion, gel, cream, lotion, ointment or serum and further comprise conventional topical/cosmetic carriers and excipients. The CTFA International Cosmetic Ingredient Dictionary and Handbook (2008), 12^{th} Edition, describes a wide variety of non-limiting cosmetic ingredients that may be used in embodiments of the present invention. Examples of these ingredient classes include but are not limited to fragrances (artificial and natural), dyes and color ingredients (e.g., Blue 1, Blue 1 Lake. Red 40, titanium dioxide, D&C blue no. 4. D&C green no. 5. D&C orange no. 4, D&C red no. 17, D&C red no. 33, D&C violet no. 2, D&C yellow no. 10, and D&C yellow no. 11), adsorbents, emulsifiers, stabilizers, lubricants, solvents, moisturizers (including, e.g., emollients, humectants, film formers, occlusive agents, and agents that affect the natural moisturization mechanisms of the skin), water-repellants, essential oils, vitamins (e.g., A, B, C, D, E. and K), trace metals (e.g., zinc, calcium and selenium), anti-irritants (e.g., steroids and non-steroidal anti-inflammatories), anti-microbial agents, chelating agents (e.g., disodium EDTA, tetrasodium EDTA, and phytic acid), preservatives (e.g., benzalkonium chloride, benzyl alcohol, phenol, urea, thimerosal, chlorobutanol, paraben such as methylparaben, propylparaben, phenoxyethanol, sodium benzoate, and potassium benzoate), pH adjusters (e.g., sodium hydroxide, hydrochloric acid, tromethamine and organic acids and bases such as citric acid), buffers (e.g. absorbents (e.g., aluminum starch octenylsuccinate, kaolin, corn starch, oat starch, cyclodextrin, tale, and zeolite), skin bleaching and lightening agents (e.g., hydroquinone and niacinamide lactate), humectants (e.g., glycerin, propylene glycol, butylene glycol, pentylene glycol, sorbitol, urea, and manitol), exfoliants (e.g., alpha-hydroxyacids, and beta-hydroxyacids such as lactic acid, glycolic acid, and salicylic acid; and salts thereof), skin conditioning agents (e.g., aloe extracts, allantoin, bisabolol, ceramides, dimethicone, hyalumnic acid, and dipotassium glycyrrhizate), sunscreen and/or sunblock compounds, dermatologically acceptable carriers, surfactants, anti-caking agents, anti foaming agents, binders, buffering agents, bulking agents, film formers or materials, e.g., polymers, for aiding the film-forming properties and substantivity of the composition, quaternary derivatives, agents increasing the substantivity, opacifying agents, thickening agents (e.g., substances which that can increase the viscosity of a composition such as carboxylic acid polymers, crosslinked polyacrylate polymers, polyacrylamide polymers, polysaccharides, and gums), and silicone containing compounds (e.g., silicone oils and polyorganosiloxanes) and combinations of the foregoing. Examples of the foregoing excipient classes can be found in U.S. Pat. Nos. 9,408,881, 10,286,030 and U.S. Patent Application Publication Nos. 2012/0076842 and 2015/0202139 that are incorporated herein by reference. The skilled artisan is aware that some excipients may be included in more than one of the foregoing classifications. Stated another way, some excipients may impart one or more properties to the topical composition.

The following provides specific non-limiting examples of some of the additional ingredients that can be used with certain embodiments of the present invention such as the cream and lotions.

Thickening/Viscosity Enhancing Agents (Including Thickeners and Gelling Agents)

The topical compositions of the present invention can comprise one or more thickening or viscosity enhancing agents. In some embodiments, one or more thickening or viscosity enhancing agent is present at a level of from about 0.05 wt % to about 15 wt %, preferably from about 0.1 wt % to about 10 wt %, and more preferably from about 0.25 wt % to about 7.5 wt %, based on the total weight of the topical composition. Nonlimiting classes of thickening or viscosity enhancing agents include those selected from the following:

a) Carboxylic Acid Polymers

The topical compositions of the present invention can optionally contain polymers that are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present invention are more fully described in U.S. Pat. Nos. 5,087,445, 4,509,949, 2,798,053, and in CTFA International Cosmetic Ingredient Dictionary. Tenth Edition, 2004.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include Ultrez® 10 (B.F. Godrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as Carbopol®1342, Carbopol® 1382, Pemulen TR-1, and Pemulen TR-2, from B.F. Goodrich. In other words, examples of carboxylic acid polymer thickeners useful herein are those selected from carbomers, acrylates/C10-C30 alkyl acrylate crosspolymers, and mixtures thereof.

b) Crosslinked Polyacrylate Polymers

The topical compositions of the present invention can optionally contain crosslinked polyacrylate polymers useful as thickeners or gelling agents including both cationic and nonionic polymers, with the cationics being generally preferred. Examples of useful crosslinked nonionic polyacrylate polymers and crosslinked cationic polyacrylate polymers are those described in U.S. Pat. Nos. 5,100,660, 4,849,484, 4,835,206. U.S. Pat. Nos. 4,628,078, 4,599,379 and EP228868.

c) Polyacrylamide Polymers

The topical compositions of the present invention can optionally contain polyacrylamide polymers, especially nonionic polyacrylamide polymers including substituted branched or unbranched polymers. Preferred among these polyacrylamide polymers is the nonionic polymer given the CTFA designation polyacrylamide and isoparaffin and laureth-7, available under the Tradename Sepigel 305 from Seppic Corporation.

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc.

d) Polysaccharides

The topical compositions of the present invention can optionally contain a wide variety of polysaccharides. “Polysaccharides” refer to gelling agents that contain a backbone of repeating sugar (i.e., carbohydrate) units. Nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation. Other useful polysaccharides include scleroglucans comprising a linear chain of (1-3) linked glucose units with a (1-6) linked glucose every three units, a commercially available example of which is Clearogel™ CS 11 from Michel Mercier Products Inc.

e) Gums

The topical compositions of the present invention can optionally contain other thickening and gelling agents which are primarily derived from natural sources. Nonlimiting examples of these thickening and gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

In certain embodiments, the topical compositions of the present invention may include a polymer or co-polymer comprising acryloyldimethyl taurate monomers, such as those described in the Jan. 14, 2016 Safety Assessment of Acryloyldimethyltaurate Polymers Used in Cosmetics, incorporated herein by reference. The polymer or co-polymer comprising acryloyldimethyl taurate monomers may be present in an amount of about 0.05 wt % to about 10 wt %, preferably from about 0.1 wt % to about 7.5 wt %, and more preferably from about 0.5 wt % to about 5.0 wt %, based on the total weight of the topical composition.

Film Forming Agents

The topical compositions of the present invention can comprise one or more film forming agents. In some embodiments, film forming agent and the thickening/viscosity enhancing agent may be the same compound. The film forming agent may be present in an amount of from about 0.05 wt % to about 50 wt %, preferably from about 0.1 wt % to about 40 wt %, and more preferably from about 0.25 wt % to about 30 wt % based on the total weight of the topical composition.

In certain embodiments, the film forming agent may be a silicone containing compound. Silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials.

The silicone containing compounds that can be used in the compositions of the present invention include silicone oils (e.g., volatile and non-volatile oils), gels, and solids. In some embodiments, the silicon containing compounds includes a silicone oil such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application. A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

The topical compositions of the present invention may also comprise one or more solvents. The solvents can include water and/or organic based solvents such as C₆ to C₂₄ branched or straight chain alkanes and/or alkenes, C₁-C₁₀ mono-alcohols (e.g., methanol, ethanol, isopropanol, etc.) C₂-C₁₂ polyalcohols (e.g. ethylene glycol, propylene glycol, hexylene glycol, glycerin, etc.) or combinations thereof. In certain embodiments, the topical compositions of the present invention comprise about 20 wt % to about 80 wt %, preferably about 30 wt % to about 75 wt % and more preferably about 40 wt % to about 65 wt % based on the total weight of the topical composition of water.

The compositions of the present invention may be prepared by any method commonly known in the industry and may include blending, mixing, emulsifying, heating, cooling steps or any combination thereof.

The topical compositions of the present invention may be packaged and dispensed in a suitable container. Containers can include a bottle, a tube (metal, plastic or laminate), a pressurized container, or pouches. The containers may include amounts of the topical composition for single or multiple applications. The containers may include indicia on its surface that instruct the subject on its application and use. The instructions for use may also be printed separately and packaged with the container comprising the topical composition.

The containers comprising multiple applications of the topical composition can dispense a pre-determined amount of the topical composition via a metered or calibrated spray, pump, or squeeze mechanism. In other embodiments, the container can be squeezed (e.g., metal, laminate, or plastic tube) to dispense a desired amount of the composition.

In certain embodiments, the subject will apply the desired amount of the topical composition to a clean and preferably dry skin surface such as the neck and décolletage area at least once a day, preferably twice or thrice a day. In certain embodiments, the subject will apply about 0.25-0.75 grams, preferably 0.30 grams to about 0.6 grams of the topical composition to the clean dry neck and décolletage area twice a day, typically once in the morning and once in the evening.

The following examples are provided by way of illustration only and are by no means intended to be limiting.

### Example 1

Topical compositions in accordance with the present invention may be prepared having the following ingredients:

| | WEIGHT PERCENT | | |
|---|---|---|---|
| INGREDIENT | Preferred | More Preferred | Most Preferred |
| Tripeptide | 0.0001-2.00 | 0.0002-1.00 | 0.0003-0.050 |
| Tetrapeptide | 0.0001-2.00 | 0.0002-1.00 | 0.0003-0.050 |
| ECM Agent | 0.001-2.00 | 0.005-1.50 | 0.007-1.00 |
| CR Agent | 0.0005-2.00 | 0.001-1.50 | 0.0025-1.00 |
| MR Agent | 0.005-2.00 | 0.01-1.50 | 0.050-1.00 |
| Antioxidant | 0.0005-4.00 | 0.0007-3.00 | 0.001-2.00 |
| Thickening Agent | 0.050-15.0 | 0.10-10.0 | 0.25-7.5 |
| Film Forming Agent | 0.050-50.0 | 0.100-40.0 | 0.25-30.0 |
| Water | 30.0-75.0 | 35.0-70.0 | 40.0-65.0 |

The topical compositions summarized in the above table may be a solution, suspension, dispersion, emulsion, gel, cream, lotion, ointment or serum and further comprise conventional topical/cosmetic carriers and excipients such as one or more emollients, chelating agents, humectants, preservatives, pH adjusting agents, buffering agents, organic solvents, surfactants, emulsifiers, sunscreen agents and combinations of the foregoing

### Example 2

Topical compositions in accordance with the present invention and particularly topical creams are prepared having the following compositions:

| INGREDIENT | WEIGHT PERCENT | | | FUNCTION |
|---|---|---|---|---|
| Water | 40-60 | 45-65 | 50-60 | solvent |
| Dimethicone | 0-35 | 10-30 | 15-25 | skin conditioning/ |
| | | | | occlusive |
| HDI-trimethylol | 0-12 | 1.5-10 | 2.5-7.5 | anticaking agent |
| crosspolymer | | | | |
| Glycerin | 0-12 | 1-10 | 2-8 | skin conditioning/ |
| | | | | humectant |
| Polysilicone-11 | 0-15 | 0.5-10 | 1-7.5 | film former |
| Butylene glycol | 0-15 | 0.5-10 | 1-7.5 | skin conditioning/ |
| | | | | solvent |
| Bis-PEG 12 dimethicone | 0-10 | 0.5-5 | 0.75-4 | skin conditioning |
| Propanediol | 0-10 | 0.25-5 | 0.5-5 | solvent |
| Sodium acrylate/sodium | 0.1-10 | 0.25-5 | 0.5-5 | film former/viscosity |
| acrylolydimethyl | | | | increasing agent |
| copolymer | | | | |
| Squalane | 0-7.5 | 0.1-5 | 0.25-5 | skin conditioning/ |
| | | | | occlusive |
| Polymethylsilsesquioxane | 0-7.5 | 0.1-5 | 0.25-5 | opacifying agent |
| Decyl glucoside | 0-5 | 0.1-3 | 0.25-2.5 | surfactant/cleansing |
| | | | | agent |
| Phenoxyethanol | 0-5 | 0.1-1 | 0.25-1 | preservative |
| Isohexadecane | 0-5 | 0.1-3 | 0.25-2.5 | skin conditioning |
| | | | | agent/emollient/solvent |
| Hydroxyacetaphenone | 0-5 | 0.1-3 | 0.25-2.5 | antioxidant |
| Physalis pubescens fruit | 0.001-3 | 0.1-2 | 0.25-1.5 | botanical |
| juice | | | | |
| Caprylic/capric | 0-3 | 0.1-2 | 0.20-1.5 | skin conditioning |
| triglyceride | | | | occlusive solvent |
| Polysorbate 80 | 0-3 | 0.1-2 | 0.20-1.5 | surfactant/emulsifying |
| | | | | agent/solubilizer |
| Acmella oleracea extract | 0.001-3 | 0.1-2 | 0.10-1.5 | botanical product |
| Silica | 0-3 | 0.1-2 | 0.10-1.5 | absorbants/dispersing |
| | | | | agent |
| Disodium EDTA | 0-3 | 0.1-2 | 0.10-1.5 | chelating agent |
| Tromethamine | 0-3 | 0.1-2 | 0.05-1.5 | pH adjuster |
| Ethylhexylglycerin | 0-3 | 0.1-2 | 0.05-1.5 | skin conditioning agent |
| Polygonum aviculare | 0.001-1 | 0.005-0.75 | 0.0075-0.05 | skin conditioning agent |
| extract | | | | |
| Chlorella vulgaris extract | 0.001-1 | 0.005-0.75 | 0.0075-0.05 | skin conditioning agent |
| Sodium citrate | 0-1.5 | 0.005-1 | 0.0075-0.75 | buffering agent |
| Hexylene glycol | 0-1.5 | 0.005-1 | 0.0075-0.75 | solvent |
| Melissa offisinalis leaf | 0.001-1 | 0.005-0.75 | 0.0075-0.05 | skin conditioning agent |
| extract | | | | |
| Lentinus edodes extract | 0.001-1 | 0.005-0.75 | 0.0075-0.05 | skin conditioning agent |
| Dunaliela salina extract | 0.001-1 | 0.005-0.75 | 0.0075-0.05 | skin conditioning agent |
| Caprylyl glycol | 0-1 | 0.001-.075 | 0.005-0.05 | preservative/skin |
| | | | | conditioning agent/ |
| | | | | emollient |
| Hydrolyzed rice protein | 0.0001-0.05 | 0.0005-0.01 | 0.0007-0.001 | skin conditioning agent |
| Potassium sorbate | 0-0.05 | 0.0001-0.01 | 0.0002-0.001 | preservative |
| Sodium benzoate | 0-0.05 | 0.0001-0.01 | 0.0002-0.001 | preservative |
| Dextran | 0-0.05 | 0.0001-0.01 | 0.0005-0.005 | binder |
| Acetyl tetrapeptide | 0.0001-0.05 | 0.0002-0.01 | 0.0003-0.005 | skin conditioning agent |
| Tocopherol | 0-0.05 | 0.0001-0.01 | 0.0002-0.001 | antioxidant |
| Phytic acid | 0-0.05 | 0.0001-0.01 | 0.0002-0.001 | chelating agent |
| Trifluoracetyl tripeptide | 0.0001-0.05 | 0.0002-0.01 | 0.0003-0.005 | skin conditioning agent |

### Example 3

In vitro studies employing a topical composition as described in Example 2 were conducted to determine the efficacy of the topical compositions of the present invention. The skin rejuvenation efficacy was assessed at a molecular biology level using quantitative real-time PCR (described in detail below). Human 3-D skin models were irradiated with ultra-violet (UV) light to mimic extrinsic skin aging prior to the application of a topical composition as described in Example 2. Specifically, an EpiDermFT™ 3D full thickness human skin model (EFT-400) from MatTek Corp. Tissues were cultured with EpiDermFT Assay Media (EFT-400-MM, MatTek Corp). EpiDermFT was irradiated with 200 mJ/cm2 ultraviolet (UV) light with UV-B filter UV lamp (Honle, Germany) to indicate an extrinsic aging model, followed by topical application of 25 μL of a composition of Example 2 or dH₂O (control), and incubated at 37° C. and 5% CO₂ for 24 hours. After incubation five tissues of each condition were placed into RNAlater® solution (ThermoFisher Scientific).

Gene expression results for UV-irradiated, non-treated tissues and UV-irradiated, Example 2-treated tissues were calculated as percentage increase compared to non-UV-irradiated, non-treated control tissues (set as 0%) after normalized with housekeeping gene GAPDH. Expression levels of genes encoding collagens (COL1A1, COL3A1, COL6A1, COL7A1 and TGFβ1), elastic fiber proteins (ELN, FBN1, FBLN5, MFAP1, LOXL-1) and the proteoglycan decorin (DCN) were assessed. The results are shown in FIGS. 1A and 1B wherein a topical composition as described in Example 2 is designated “NCC”. Application of a topical composition as described in Example 2 resulted in significant upregulation of all genes tested compared to UV-irradiated, non-treated samples indicating that the topical compositions of Example 2 stimulate ECM components for dermal structure improvement that may translate into firmer skin, as well as improvement of the dermal-epidermal junction (DEJ) which is important for skin elasticity. Additionally, elastase activity assays showed that the topical compositions of Example 2 significantly reduced the level of elastase, (an enzyme that breaks down elastin) following UV irradiation suggesting protection against elastic fiber degradation (FIG. 1C).

Aging alters essential processes involved in maintaining cellular proteostasis (protein homeostasis), including the proteasome and the autophagy systems, leading to an accumulation in cellular debris, protein aggregation, and cellular damage. As shown in FIGS. 2A-2C, gene expression levels of key recycling genes involved in proteasome activity (POMP, PSMB5 and PSMB6) and autophagy (ATG5, ATG7, ATG12 and BECN1) along with a heat shock protein that facilitates and stabilizes proper folding of proteins (HSPA8) were increased with a topical composition as described in Example 2 (designated as “NCC”), indicating stimulation of the various cellular recycling processes.

The in vitro pre-clinical data demonstrates the efficacy of the topical compositions of the present invention and particularly the compositions of Example 2, in providing overall skin rejuvenation benefits by boosting various ECM and DEJ components as well as supporting systems that help maintain proteostasis.

Quantitative Real-Time PCR

mRNA was extracted from the human skin model tissues (Maxwell® RSC simplyRNS Tissue Kit; Promega) followed by cDNA synthesis (High-Capacity cDNA Reverse Transcription Kit; ThermoFisher Scientific) and quantitative real-time PCR (TaqMan Fast Advanced Master Mix, ThermoFisher Scientific). Gene expression analyses were performed using TaqMan Gene Expression Assays (ThermoFisher Scientific) with real-time PCR system QuantStudio7 Flex (ThermoFisher Scientific). The assay mix used for studies were: GAPDH (Cat #: Hs02758991_g1), COL1A1 (Cat #: Hs001640004_m1); COL3A1 (Cat #: Hs00943809_m1), COL6A1 (Cat #: Hs01095585_m1), COL7A1 (Cat #: Hs00164310_m1), TGFB1 (Cat #: Hs00998133_m1), DCN (Cat #: Hs00754870_s1), ELN (Cat #: Hs00355783_m1), FBN1 (Cat #Hs00171191_m1), FBLN5 (Cat #: Hs00197064_m1), MFAP1 (Cat #: Hs00195537_m1), LOXL1 (Cat #Hs00935937_m1), POMP (Cat #: Hs01106088_m1), PSMB5 (Cat #: Hs00605652_m1), PSMB6 (Cat #: Hs00382586_m1), ATG5 (Cat #: Hs00169468_m1), ATG7 (Cat #: Hs00893766_m1), ATG12 (Cat #: Hs04980076_s1), BECN1 (Cat #: Hs01007018_m1). HSPA8 (Cat #: Hs03044880_gH)

Elastase Assay

The assay employed was based on methods previously described in literature. In brief, human skin model tissue was homogenized and solubilized in 0.1% Triton-X 100, 0.2 M Tris-HCl (pH 8.0) buffer, followed by ultrasonication and subsequent centrifugation at 2000 g for 10 min to obtain supernatants for enzyme assay. To measure elastase activity, 2 μL of substrate (62.5 mM, N-succinyl-tri-alanyl-p nitroanilide from Sigma-Aldrich) was incubated with 100 μL lysate solution at 37° C. for 2 h. The amount of released nitroaniline was measured by determining absorbance at 410 nm using a spectrophotometer (Molecular Devices).

### Example 4

A randomized, double-blind, regimen-controlled study was conducted to assess the efficacy and tolerability of the topical compositions of Example 2 as a topical treatment for the neck and the décolleté area. Criteria for study participation included female subjects aged 45-70 years, presenting moderate to severe overall skin texture on the neck and moderate to severe skin tone unevenness on the décolleté.

Institutional Review Board approval (IntegReview IRB, Austin, Tex.) was obtained prior to conduct of any study procedures. The conduct of the study followed all applicable guidelines for the protection of human subjects for research as per 21 CFR 50, in accordance with accepted standards for Good Clinical Practices (GCP) and International Conference on Harmonization (ICH). All subjects provided informed consent prior to study participation.

Subjects were not allowed to apply any other topical products than the ones provided (including skin brighteners, retinoids, alpha/beta/poly-hydroxy acids) nor undergo treatments to the test area. All subjects were randomized either to the NCC-treatment group which used a topical composition as described in Example 2 twice-daily (once in the morning and night, after cleansing) in addition to standard skin care products (SkinMedica® Facial Cleanser and SkinMedica® Essential Defense Mineral Shield SPF 35 Sunscreen), or to the control group, which only used the provided standard skin care products. All subjects were provided with pre-weighed units of assigned products of which they were instructed to apply a thin layer to the neck and décolletage and blend in an upward motion. A total of 75 subjects were enrolled in the study with 46 subjects in NCC-treatment group and 29 subjects in control group.

Clinical Efficacy Assessment

Clinical efficacy was conducted at baseline, weeks 4, 8 and 12 by the investigator who was blinded to the treatment randomization of the subject. Subjects were instructed to cleanse their neck, face and décolleté and remove all makeup at least 30 minutes prior to each scheduled visit. Clinical assessments and standardized photographs of the neck and décolleté were taken at each visit. Assessments of efficacy parameters were conducted at all study visits using a modified Griffiths' 10-point grading scale, where 0=none (best possible condition), 1-3=mild, 4-6=moderate, and 7-9=severe (worst condition possible), with half-point scores assigned as necessary to accurately describe the skin condition. Crepiness, laxity (sagging), and overall skin texture were only assessed on the neck and not décolletage. Efficacy parameters are listed below with description of grade 0 and 9 anchors:

Fine lines/wrinkles: 0=None, 9=Numerous, long, deep fine lines/wrinkles

Coarse lines/wrinkles: 0=None, 9=Numerous, long, deep coarse lines/wrinkles

Crepiness: 0=appears smooth with no wrinkles or “crinkliness”, 9=Skin appears thin and easily crinkled when lightly pinched

Tactile roughness/smoothness: 0=Smooth, even feeling skin texture, 9=Rough, uneven feeling skin texture

Laxity (sagging): 0=Firm, dense appearance, no sagging, 9=Soft, droopy appearance, prominent skin folds

Overall skin texture: 0=Smooth skin appearance and touch no roughness, laxity, nor crepey/crinkled appearance, 9=Pronounced, extensive visible skin roughness, lines/wrinkles, laxity and crepey/crinkled appearance

Hyperpigmentation: 0=Even skin color, no observable hyperpigmentation, 9=Significant (severe) hyperpigmented appearance, involving most of the area, with very strong intensity

Skin tone evenness: 0=Even, healthy skin color, 9=Uneven, extensive discolored appearance (brown and red colors)

Radiance/luminosity/brightness: 0=Radiant, luminous or glowing appearance, 9=Dull/matte and/or sallow skin appearance

In addition, at weeks 4, 8, and 12, an expert grader assessed the global improvement in overall skin texture (pronounced, extensive visible skin roughness, lines/wrinkles, laxity and crepey/crinkled appearance) on the neck and overall photodamage on the décolleté using a grading scale, where 1=worse, 2=no improvement, 3=mildly improved, 4=moderately improved, and 5=markedly improved.

Standardized Photography

Stephens & Associates Photo Station Imaging

Digital images were taken of each subject's face, neck, and décolleté (center view, left side, and right side) using Stephens & Associates photo station with a Canon Mark II 7D digital SLR camera (Canon Inc., Tokyo, Japan) with a Canon EF-S 60 mm f/2.8 macro lens under visible light. Unfiltered full-spectrum (white) light was provided using Profoto D1 (500W) studio strobes affixed to the photo station. Canon EOS utility software was used for image overlay to ensure post-baseline images match baseline images. Prior to photography, subjects were instructed to remove any jewelry from the area to be photographed, cleansed their face and equilibrated in the clinic for at least fifteen minutes.

Cutometer Measurements

A single measurement was taken of each subject's left side of the neck, in the center (vertically), approximately left of the laryngeal prominence (“Adam's apple”) using Cutometer MPA 580 (Courage+Khazaka electronic GmbH, Köln, Germany) to measure the viscoelastic properties (ie, firmness and elasticity) of the skin. For each subject, the measured location was marked on a body diagram to ensure the same site was measured at week 12.

Subject Self-Assessment Questionnaire

Subjects completed a self-assessment questionnaire regarding various skin parameters and product texture and attributes at weeks 4, 8, and 12.

Statistical Analysis

Clinical grading scores at weeks 4, 8, and 12 were compared to baseline scores using Wilcoxon signed-rank test and comparisons between treatment groups using Wilcoxon rank sum test. Average percent change from baseline/placebo was calculated for all parameters at each follow-up visit. Comparisons between treatment groups were calculated for all parameters at each follow-up visit. All differences are considered statistically significant at the p≤0.05 level.

Sixty-nine female subjects, aged 45-70 with Fitzpatrick Skin Types I-V, completed the twelve week, double-blind, randomized, regimen-controlled study (NCC-treatment group: n=42, control group: n=27). Overall results indicate that twice daily topical application of topical compositions in accordance with the present invention and particularly the topical compositions of Example 2 were effective in improving neck and décolletage skin condition and firmness when used in combination with a sunscreen over the course of 12 weeks.

For the neck, the topical compositions of Example 2 provide statistically significant improvements in the appearance of neck Sagging/Skin Laxity at as early as 8 weeks with continued improvements through week 12, when compared to control (all p≤0.01; Wilcoxon rank sum test) as well as when compared to baseline (all p≤0.01; Wilcoxon signed rank test) (FIG. 3A). The topical compositions of Example 2 also provide significant improvements in Fine Lines/Wrinkles, Tactile Roughness/Smoothness, and Crepiness at all follow-up visits and in Coarse Lines/Wrinkles, Hyperpigmentation, Skin Tone Evenness, Radiance, Laxity/Sagging and Overall Skin Texture at weeks 8 and 12, when compared to baseline (all p≤0.035; Wilcoxon signed rank test), (FIG. 3B).

For the décolletage, the topical compositions of Example 2 provide significant improvements in fine lines/wrinkles and tactile roughness/smoothness at all follow-up visits, and in coarse lines/wrinkles, hyperpigmentation, skin tone evenness, and radiance at weeks 8 and 12, when compared to baseline (all p≤0.035; Wilcoxon signed rank test) (FIG. 3C).

Investigator's global improvement assessment showed significant improvements over control at weeks 8 and 12 in neck skin laxity/sagging and overall neck skin texture (which includes pronounced, extensive visible skin roughness, lines/wrinkles, laxity and crepey/crinkled appearance) (p≤0.009; Wilcoxon rank sum test). Compared to baseline, the NCC-treatment group showed significant global improvements in overall skin texture on the neck and in overall photodamage on the décolletage at all follow-up visits (all p≤0.002; Wilcoxon signed rank test). Improvements were observed in the control group but did not reach statistical significance until week 12 for neck skin texture (p≤0.016; Wilcoxon signed rank test), and week 8 for overall photodamage on the décolletage (p≤0.031; Wilcoxon signed rank test).

Three subjects reported potential treatment-related mild/moderate adverse events (erythema/rash/burning sensation): two subjects in the NCC treatment group and one subject in the control group. The adverse events (AE) all resolved without sequelae. One of the subjects in the NCC treatment group discontinued due to the AE. Cutometer measurements demonstrated statistically significant improvements in extensibility (decreased extensibility indicates reduced ability of skin to be extended or stretched), maximum recovery area (decreases with increased firmness of the skin), resiliency, pure elasticity, total recovery (overall elasticity), and elastic recovery for the NCC-treatment group at week 12 when compared with baseline (all p≤0.035; Wilcoxon signed rank test). NCC-treatment group showed consistently greater improvements numerically over control. The control group showed statistically significant improvements (i.e. decrease in measurement) in extensibility and maximum recovery area at week 12, while biological elasticity and viscoelastic recovery did not change for either group (FIG. 3D). Altogether, these results indicate an overall increase in skin elasticity and firmness.

Subject self-assessment questionnaires support the investigator-observed improvements in neck and décolletage with a significant proportion of subjects agreeing that the topical compositions of Example 2 made their neck look and feel firmer, reduced the appearance of lines and wrinkles, and improved skin texture at all follow-up visits (all p≤0.007; binomial test). Furthermore, the attributes of the topical compositions of Example 2 were highly rated by subjects. 100% of subjects approved the texture and odor, ease of application, skin absorption as well as compatibility and convenience for use as part of a daily skincare regimen.

The topical compositions of the present invention and particularly the topical creams described in Example 2 were clinically proven to be effective and well-tolerated in significantly improving multiple key skin quality parameters of the neck and décolletage including sagging/skin laxity, crepiness and skin tone evenness, supported by blinded investigator assessments as well as objective instrumentation measurements. The in vitro data presented in Example 3 and FIGS. 1A-1C and 2A-2C demonstrates that the topical composition of the present invention induces the gene expression of various key ECM components as well as reduces the levels of elastase. Collagens type VII (COL7A1) is one of the key protein present in the basement membrane of the DEJ while (ELN, FBN1, FBLN5, MFAP1, LOXL-1) govern the formation of healthy and functional elastic fibers. Collagens type I and III (COL1A1 and COL3A1) constitute most of the dermis providing skin its firmness and strength for a more youthful appearance, and decorin (DCN) is a proteoglycan important for the proper alignment of these fibers. Collagen type VI (COL6A1) is one of the major components of skeletal muscle ECM and thus, is involved in health and function of muscle fibers. Supporting COL6A1 expression can be of particular relevance to improving the overall appearance of aged neck skin since accentuation of platysma muscle protrusion and platysmal bands on the neck mid-line could be tied to weakening and loss of tissue and fibers that connect muscle to the overlying skin.

The proteasome and autophagy systems are part of the cellular recycling mechanism that ensures orderly degradation of damaged, dysfunctional and misfolded proteins and cellular components to maintain cellular proteostasis. Aging alters the activity of these systems leading to accumulation of damaged cellular components and an exacerbated intrinsic aging process. The topical compositions of Example 2 result in upregulation of various genes related to proteasome assembly and function (POMP, PSMB5 and PSMB6), different stages of autophagy (ATG5, ATG7, ATG12 and BECN1), and protein folding and stabilization (HSPA8) suggesting that the compositions help maintain proteostasis and mitigate intrinsic aging processes to support a healthier and more youthful appearance of the neck and décolletage.

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A topical composition comprising: (i) one or more tripeptides, one or more tetrapeptide or a combination thereof and (ii) one or more botanical extracts selected from the group consisting of extracellular matrix (“ECM”) agents; cellular recycling (“CR”) agents; muscle contracting (“MR”) agents; antioxidants or combinations thereof.

2. The topical composition as defined in claim 1 wherein the composition comprises about 0.0001 wt % to about 2.00 wt % based on the total weight of the composition of a tripeptide.

3. The topical composition as defined in claim 1 wherein the composition comprises about 0.0001 wt % to about 2.00 wt % based on the total weight of the composition of a tetrapeptide or a combination thereof.

4. The topical composition as defined in claim 1 comprising one or more ECM agents selected from the groups consisting of Chlorella vulgaris extract, Lentinus edodes extract or a combination thereof.

5. The topical composition as defined in claim 1 comprising one or more CR agents selected from the group consisting of Mellissa officinalis leaf extract, rice extract or a combination thereof.

6. The topical composition as defined in claim 1 comprising Acmella oleacera extract.

7. The topical composition as defined in claim 1 comprising one or more antioxidants selected from the group consisting of Physalis pubescens fruit juice, Dunaliella salina extract, Polygonum aviculare extract or combinations thereof.

8. The topical composition as defined in claim 1 further comprising a thickening agent.

9. The topical composition as defined in claim 8 wherein the thickening agent comprises a polymer or copolymer with acryloyldimethyl taurate monomers.

10. The topical composition as defined in claim 9 wherein the thickening agent is a sodium acrylate-sodiumacryloldimethyl copolymer.

11. The topical composition as defined in claim 1 further comprising a film forming agent.

12. The topical composition as defined in claim 11 wherein the film forming agent comprises a silicone containing compound.

13. The topical compositions as defined in claim 12 wherein the silicone containing compound is a polyorganosiloxane.

14. A method of treating aging skin of the neck and décolletage area comprising applying the topical composition as defined in claim 1 to a subject's neck and décolletage area at least once a day.

15. The method defined in claim 15 wherein the topical composition is applied at to the subject's neck and décolletage area twice a day.
